(19) United States
(12) Patent Application Publication
(10) Pub. No.: US 2022/0087872 A1
(43) Pub. Date: Mar. 24, 2022

(54) Nursing Pads with Straps

(71) Applicant: Damaris Andrade, Cornelius, OR (US)

(72) Inventor: Damaris Andrade, Cornelius, OR (US)

(73) Assignee: Damaris Andrade, Cornelius (US)

(21) Appl. No.: 17/140,669

(22) Filed: Jan. 4, 2021

Related U.S. Application Data
(60) Provisional application No. 63/082,141, filed on Sep. 23, 2020.

(51) Int. Cl. A61F 13/14

(52) U.S. Cl. CPC A61F 13/141; A61F 2013/15016

(57) ABSTRACT

A nursing pad apparatus is constructed according to a method starting with constructing a top layer and a bottom layer from one or more pieces of fabric and an interior set of layers comprised of absorbent material to be placed between the top and bottom layers. The top and bottom layers are combined via a fabric edge, creating a void space for the interior set of layers. A fabric strap is affixed at the edge of the nursing pad apparatus and a fastener is positioned on the first and second ends of the fabric strap. The fabric strap is used to couple the nursing pad apparatus to an article of clothing, with the fasteners used to temporarily affix the nursing pad apparatus in place. The fasteners consist of snaps, eyes hooks, and button and loop.

### CROSS-REFERENCE TO RELATED APPLICATIONS

This application claims the benefit of U.S. provisional patent application Ser. No. 63/082,141, filed on 23 Sep. 2020 by Andrade, and entitled NURSING PADS WITH STRAPS, the entire disclosure of which is incorporated by reference herein.

### BACKGROUND

The present disclosure relates generally to nursing pads. In particular, nursing pads with straps are described.

Nursing pads are protective layers designed to absorb and catch leaking breast milk while being worn by lactating women. The nursing pads are generally placed between the woman's breast and the inside of her bra. The nursing pads also protect the woman's bra and clothing from becoming wet or stained from leakage

Known nursing pads are often bulky, uncomfortable, and may be seen through clothing or dropped while inserting or removing it from the bra. Many nursing pads are fully removable, creating unhygienic conditions if the pad is accidentally dropped, or needing to be place on a surface to free the hands, or if the used nursing pad is touched. The nursing pad may also shift, fold, crumple, or bunch while worn. Not only is this uncomfortable, but the nursing pad is likely to be seen through the woman's clothing.

Thus, there exists a need for nursing pads that improve upon and advance the design of known nursing pads. Examples of new and useful nursing pads relevant to the needs existing in the field are discussed below.

### SUMMARY

Disclosed herein are embodiments of a nursing pad apparatus, a method for coupling the nursing pad apparatus to an article of clothing, and a method for constructing a nursing pad apparatus.

A nursing pad apparatus comprises a top surface layer comprised of a first one or more pieces of fabric. The nursing pad apparatus further includes a bottom surface layer comprised of a second one or more pieces of fabric. The nursing pad apparatus further includes a set of interior layers between the top and bottom surfaces, the set comprised of an absorbent material. The nursing pad apparatus further includes an edge comprised of a third one or more pieces of fabric, wherein the edge joins the top surface layer and the bottom surface layer to create a space. The space contains the set of interior layers.

A method for coupling a nursing pad apparatus with a fabric strap to an article of clothing may begin with looping the fabric strap of the nursing pad apparatus around a part of an article of clothing. A first end of a fastener on the fabric strap is attached to a second end of the fastener on the edge.

A method for constructing a nursing pad apparatus with a fabric strap may begin with positioning a set of interior layers between a top surface layer and a bottom surface layer. The top surface layer and the bottom surface layer are combined via an edge. A first end of the fabric strap is affixed at a first position on the edge. A first end of a fastener is affixed at a second position on the edge. At the second end of the fabric strap, on or more second ends of the fastener are affixed.

The above summary is not intended to describe each illustrated embodiment or every implementation of the present disclosure.

### BRIEF DESCRIPTION OF THE DRAWINGS

The drawings included in the present disclosure are incorporated into, and form part of, the specification. They illustrate embodiments of the present disclosure and, along with the description, serve to explain the principles of the disclosure. The drawings are only illustrative of typical embodiments and do not limit the disclosure.

FIG. 1 is a top view of a first example of a nursing pad, according to embodiments.

FIG. 2 is a bottom view of the nursing pad shown in FIG. 1, according to embodiments.

FIG. 3 is a cross-sectional view of the nursing pad shown in FIG. 1, according to embodiments.

While the embodiments described herein are amenable to various modifications and alternative forms, specifics thereof have been shown by way of example in the drawings and will be described in detail. It should be understood, however, that the particular embodiments described are not to be taken in a limiting sense. On the contrary, the intention is to cover all modifications, equivalents, and alternatives falling within the spirit and scope of the invention.

### DETAILED DESCRIPTION

Aspects of the present disclosure relate to nursing pads, and more particularly to nursing pads with straps. While the present disclosure is not necessarily limited to such applications, various aspects of the disclosure may be appreciated through a discussion of various examples using this context.

The disclosed nursing pads will become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

Throughout the following detailed description, a variety of nursing pad examples are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

### Definitions

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional elements or method steps not expressly recited.

Terms such as “first,” “second,” and “third” are used to distinguish or identify various members of a group, or the like, and are not intended to denote a serial, chronological, or numerical limitation.

“Coupled” means connected, either permanently or releasably, whether directly or indirectly through intervening components.

Nursing Pads with Straps

With reference to the figures, Nursing Pads With Straps will now be described. The nursing pads discussed herein function to limit movement of the nursing pad and increase the comfort of the user while worn, and create a more hygienic process of inserting, adjusting, and removing the nursing pad. A strap attached to the nursing pad secures to an article of clothing, such a bra or tank top. Material for the exterior of the nursing pad and strap may be soft such as bias sating tate. The reader will appreciate from the figures and description below that the presently disclosed nursing pads address many of the shortcomings of conventional nursing pads.

### Nursing Pad Embodiment One

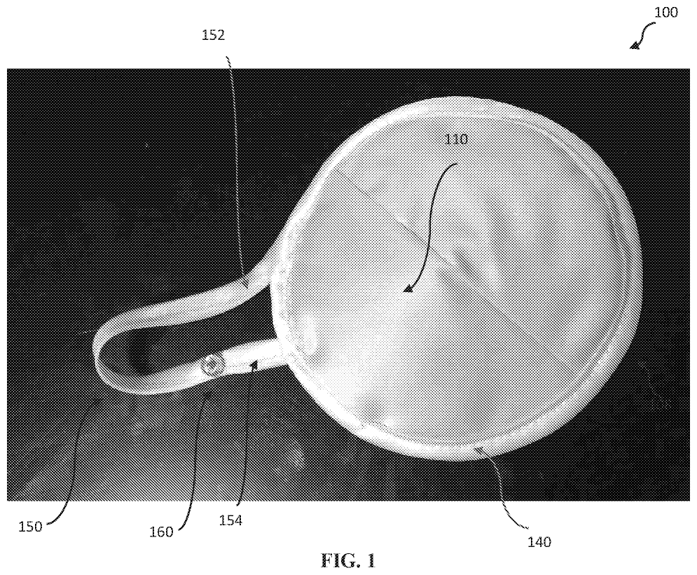

With reference to FIGS. 1-3, a first example of a nursing pad, nursing pad 100, will now be described. Nursing pad 100 includes a top surface 110, a bottom surface 120, interior layers 130, an edge 140, a strap 150, a first end 152, a second end 154, and fasteners 160.

In some examples, the nursing pad does not include one or more features included in nursing pad 100. For example, some nursing pads 100 examples do not contain edge 140.

In other examples, the nursing pad 100 includes additional or alternative features, such as additional straps or more than one type of fastener on the nursing pad 100.

Top Surface 110

As can be seen in FIG. 1, the top surface 110 of the nursing pad 100 is contoured. This shape allows the nursing pad to comfortably fit over the user's breast. Multiple sizes of the nursing pad 100 may be provided to accommodate different breast sizes. The top surface 110 may be made from many types of material, such as organic bamboo. In one embodiment, the material is waterproof to prevent leakage of the breast milk onto the user's bra and/or clothing. In one embodiment, a smooth material is used to reduce the likelihood of the nursing pad 100 showing through the bra and/or clothing. In one embodiment, a textured material is used to cling to the user's bra or clothing to prevent slippage of the nursing pad 100 while worn. The top surface 110 may be made from one piece of material or multiple pieces. The color of the top surface 110 may be a light color, such as white or cream, to minimize the likelihood of being seen through the bra and/or clothing. Other colors may also be used to match the user's bra and/or clothing to prevent the nursing pad 100 from showing.

Bottom Surface 120

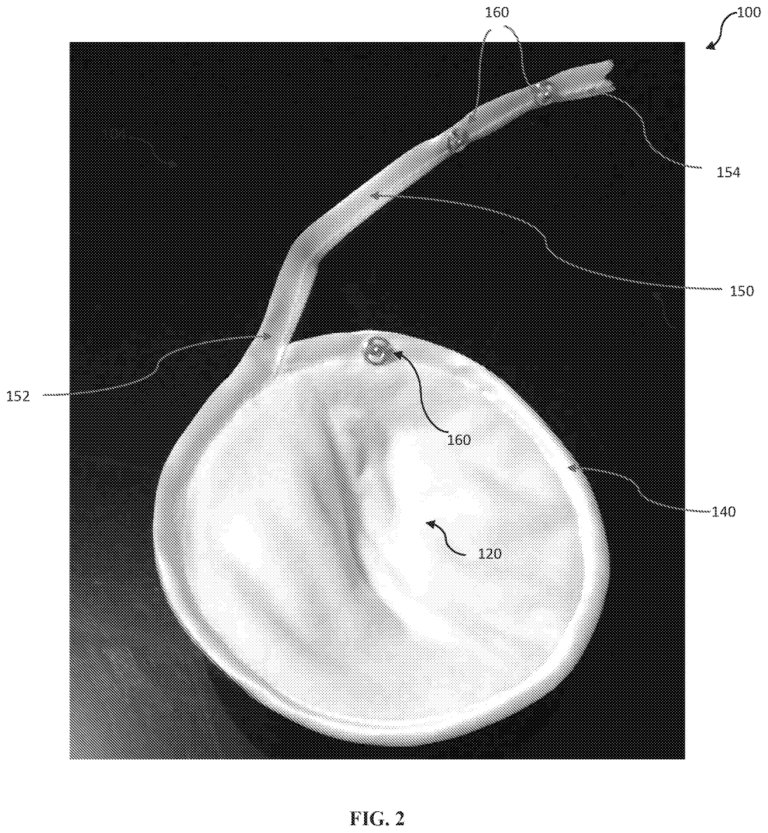

In the example shown in FIG. 2, the bottom surface 120 of the nursing pad 100 is contoured similarly to the top surface 110. The bottom surface 120 may be made from many types of material, such as material that is breathable. In one embodiments, a soft and/or smooth material is used to reduce the likelihood of chafing. The bottom surface 120 may be made from one piece of material or multiple pieces. The color of the bottom surface 120 may be any color. In one embodiment, the color of the bottom surface 120 is the same as the top surface 110 to make it more aesthetically pleasing. In one embodiment, the color of the bottom layer 120 may be a light color to provide a visual indicator of problems with the breast milk. Other colors may also be used to match the user's bra and/or clothing to prevent the nursing pad 100 from showing.

Interior Layers 130

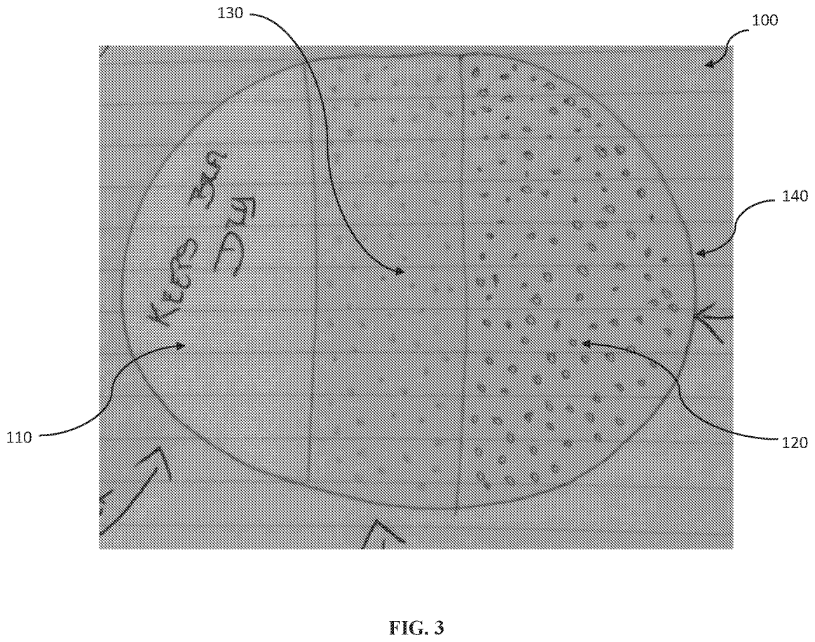

In the example shown in FIG. 3, the interior layers 130 are located between the top surface 110 and the bottom surface 120 and similarly contoured. The interior layers 130 function to absorb the leaking breast milk. The interior layers 130 may be an absorbent, microfiber material. In one embodiment, the interior layers 130 include a waterproof layer and an absorbent layer. This configuration may provide an additional layer of waterproof protection or allow for the top surface 110 to be a non-waterproof material, such as something softer or more tactilely pleasing. In one embodiment, the interior layers 130 include two absorbent layers, wherein a first layer is of a similar size to the top surface 110 and bottom surface 120 and a second layer is smaller than the first layer and covers the nipple area of the user.

Edge 140

In the example shown in FIGS. 1-2, the edge 140 combines edges of the top surface 110 and the bottom surface 120, leaving a hollow space between for the internal layers 130 to reside. The edge 140 may be made from many types of material, such as bias sating tate. In one embodiment, the material is waterproof to prevent leakage of the breast milk onto the user's bra and/or clothing. In one embodiment, a smooth material is used to reduce the likelihood of the nursing pad 100 showing through the bra and/or clothing. In one embodiment, a smooth material is used to reduce the likelihood of the nursing pad 100 showing through the bra and/or clothing. In one embodiment, a textured material is used to cling to the user's bra and/or clothing to prevent slippage of the nursing pad 100 while worn. In one embodiment, a soft and/or smooth material is used to reduce the likelihood of chafing. The edge 140 may be made from one piece of material or multiple pieces. The color of the edge 140 may be a light color, such as white or cream, to minimize the likelihood of being seen through the bra and/or clothing. In one embodiment, the color of the edge 140 is the same as the top surface 110 and/or bottom surface 120. Other colors may also be used to match the user's bra and/or clothing to prevent the nursing pad 100 from showing.

Strap 150

In the example shown in FIGS. 1-2, the strap 150 functions to maintain the position of the nursing pad 100 over the user's breast and prevent the movement of the nursing pad 100 while the user is wearing it. The strap 150 includes the first end 152 and the second end 154. The first end 152 is attached to the nursing pad 100 and the second end 154 includes the fasteners 160. In one embodiment, the first end 152 is attached to the edge 140. The strap 150 may be long enough to loop around a part of the user's clothing, such as a bra. In one embodiment, the second end 154 is attached to the nursing pad 100 via the fasteners 160. The edge 140 may contain multiple fastener receptacles (buttonhole, snap bottom, hook, etc.). The receptacles may be placed all around the edge 140 for maximum configurations. For example, a receptacle may be placed adjacent to the area of attachment of the first end 152 to the nursing pad 100 to create a narrow loop of the strap 150. In another example, a receptacle may be placed further away to create a wider loop of the strap 150. The second strap functions to either attach to another part of the bra or allow for tighter cinching near the location of the strap 150.

The strap 150 also functions to provide the user a hygienic way to touch the nursing pad 100. The user may only need to tough the strap 150 to remove the nursing pad 100, such as by pulling on the strap. This helps to prevent dropping of the pad, as it is already secured by the strap 150.

Many types of material may be used for the strap 150, such as bias satin tate. In one embodiment, a smooth material is used to reduce the likelihood of the nursing pad 100 showing through the bra and/or clothing. In one embodiment, a textured material is used to cling to the user's bra or clothing to prevent slippage of the nursing pad 100 while worn. In one embodiment, a soft and/or smooth material is used to reduce the likelihood of chafing. The strap 150 may be made from one piece of material or multiple pieces. The color of the strap 150 may be a light color, such as white or cream, to minimize the likelihood of being seen through the bra and/or clothing. In one embodiment, the color of the strap 150 is the same as the top surface 110, the bottom surface 120, and/or the edge 140. Other colors may also be used to match the user's bra and/or clothing to prevent the nursing pad 100 from showing.

Fasteners 160

In the example shown in FIGS. 1-2, the fasteners 160 are located on the second end 154 of the strap 150 and the edge 140. As described above, the fasteners 160 may be snaps, hook and eye, buttons with a corresponding buttonhole, or other means of detachably connecting the strap 150 to the nursing pad 100. The fasteners 160 function to removably attach the second end 154 of the strap 150 to the nursing pad 100, sometimes specifically on the edge 140. In one embodiment, two fasteners 160 in the forms of snaps are located on the second end 154 and the edge 140. The male end of the snap is located on the second end 152 and the female end, or receptacle as described above, on the edge 140. IN one embodiment, the snaps may be plastic as to be more comfortable for the user. In one example, fasteners 160 are located on the first end 152 and the second end 154. This allows the strap to be completely removed from the nursing pad 100.

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

What is claimed is:

1. A nursing pad apparatus, comprising:
a top surface layer comprised of a first one or more pieces of fabric;
a bottom surface layer comprised of a second one or more pieces of fabric;
a set of interior layers between the top and bottom surfaces, the set comprised of an absorbent material; and
an edge comprised of a third one or more pieces of fabric,
wherein the edge joins the top surface layer and the bottom surface layer to create a space; and
wherein the space contains the set of interior layers.

2. The nursing pad apparatus of claim 1, further comprising:
a fabric strap, the fabric strap including a first end and a second end; and
wherein the first end is affixed to the edge.

3. The nursing pad apparatus of claim 2, wherein a first end of a fastener is affixed on the edge and a second end of the fastener is affixed to the fabric strap.

4. The nursing pad apparatus of claim 3, wherein the second end of the fabric strap is removably attached to the edge with the second end of the fastener.

5. The nursing pad apparatus of claim 3, wherein the first end of the fastener is a male end and the second end of the fastener is a female end.

6. The nursing pad apparatus of claim 3, wherein the first end of the fastener is a female end and the second end of the fastener is a male end.

7. The nursing pad apparatus of claim 5, wherein the female end of the fastener includes a set of female ends spaced along the second end of the fabric strap.

8. The nursing pad apparatus of claim 6, wherein the male end of the fastener includes a set of male ends spaced along the second end of the fabric strap.

9. The nursing pad apparatus of claim 3, wherein the fastener is selected from the group consisting of snaps, eyes and hooks, and buttons.

10. A method for coupling a nursing pad apparatus with a fabric strap to an article of clothing, comprising:
looping the fabric strap of the nursing pad apparatus around a part of an article of clothing; and
attaching a first end of a fastener on the fabric strap to a second end of the fastener on the edge.

11. The method of claim 10, wherein the first end of the fastener is a male end and the second end of the fastener is a female end.

12. The method of claim 10, wherein the first end of the fastener is a female end and the second end of the fastener is a male end.

13. A method for constructing a nursing pad apparatus with a fabric strap, comprising:
positioning a set of interior layers between a top surface layer and a bottom surface layer;
combining, via an edge, the top surface layer and the bottom surface layer;
affixing, at a first position on the edge, a first end of the fabric strap;
affixing, at a second position on the edge, a first end of a fastener; and
affixing, at a second end of the fabric strap, one or more second ends of the fastener.

14. The method of claim 13, wherein the top and bottom surface layers are comprised of breathable fabric.

15. The method of claim 13, wherein the top and bottom surface layers are conical in shape.

16. The method of claim 13, wherein the set of interior layers is comprised of absorbent fabric.

17. The method of claim 13, wherein the first end of the fastener is a male end and the second end of the fastener is a female end.

18. The method of claim 13, wherein the first end of the fastener is a female end and the second end of the fastener is a male end.

19. The method of claim 13, wherein the fastener is selected from the group consisting of snaps, eyes and hooks, and buttons.
